(19) United States
(12) Patent Application Publication
(10) Pub. No.: US 2025/0206697 A1
(43) Pub. Date: Jun. 26, 2025

(54) PROCESS FOR THE PRODUCTION OF A SURFACTANT

(71) Applicant: BASF SE, Ludwigshafen Am Rhein (DE)

(72) Inventors: Sylvester GROESSL, Ludwigshafen (DE); Juergen TROPSCH, Ludwigshafen (DE); Claudia BRUNN, Duesseldorf-Holthausen (DE); Dagmar Pascale KUNSMANN-KEITEL, Ludwigshafen (DE); Sandra HOMANN, Ludwigshafen (DE)

(21) Appl. No.: 18/849,055

(22) PCT Filed: Mar. 30, 2023
(86) PCT No.: PCT/EP23/58273

§ 371 Date: Sep. 20, 2024

(30) Foreign Application Priority Data
Mar. 30, 2022 (EP) 22165305.8

(51) Int. Cl. C07C 227/18; B01J 23/44; C11D 1/88

(52) U.S. Cl. CPC C07C 227/18; B01J 23/44; C11D 1/88

(57) ABSTRACT

Described herein are a process for the production of a surfactant of formula (I), as well as a surfactant composition. Also described herein are specific surfactants and compositions thereof, as well as methods of their use in a wide variety of applications such as all purpose cleaning agents.

The present invention relates to a process for the production of a surfactant. The invention further relates to individual embodiments of the surfactant, as well as surfactant compositions.

Surfactants are compounds which lower the surface tension between two phases, in particular between two liquids. Surfactants generally are organic amphiphilic compounds, i.e., compounds comprising both hydrophobic and hydrophilic groups. Such compounds find use, e.g., as detergents, wetting agents, foaming agents, emulsifiers, and dispersants.

N-alkyl amino acid-based sur0factants have shown promise as amphoteric surfactants. These surfactants have amino acids as polar zwitterionic headgroups. N-alkyl amino acid-based surfactants exhibit low critical micelle concentrations (CMCs) and provide low values of surface tension at the CMC.

Being commodities, surfactants are produced on a large scale, and there is a pressing need to develop direct sustainable catalytic methods to obtain environmentally friendly and fully bio-based alternatives, especially in the context of a bio-based economy. Despite the obvious potential of N-alkyl amino acids, there is a relative lack of selective catalytic methods to obtain these compounds via direct functionalization of unprotected amino compounds such as α-amino acids.

In general, N-alkylation of α-amino acids has been performed using stoichiometric methods, such as reductive amination of aldehydes with complexing salts, or nucleophilic substitution with alkyl halides. One method described in the art comprises reductive amination of fatty aldehydes with amino acids in the presence of elemental hydrogen.

Several publications describe the reductive amination of dodecanal with L-proline or L-hydroxyproline in the presence of palladium on carbon (Pd/C), in an alcohol, at room temperature and at a hydrogen pressure of 1 bar, including Yan et al., J. Sep. Sci. 2017, 40, 1834-1842, Tanimura et al., J. Chromatogr. 1984, 284, 285-288, Tanimura et al., Anal. Chem. 1984, 56, 1152-1155, Tong et al., J. Sep. Sci. 2018, 41, 1479-1488, and Cussler et al., Am. Inst. Chem. Eng. 1992, 38, 10, 1493-1498.

Joly et al., J. Chem. Soc. Perkin Trans. 2 2001, 998-1004, describe the reductive amination of dodecanal with L-proline or L-hydroxyproline in the presence of Pd/C and in an alcohol solvent at room temperature and a hydrogen pressure of 50 bar.

US 2019/0218170 A1 describes branched trialkylamine precursors for the production of surfactants. Numerous starting materials including 2-ethylhexanal and sarcosine, and a broad range of conditions for obtaining the precursors are described.

FR 2 469 395 A1 mentions the reductive amination of dodecanal with pipecolic acid in the presence of Pd/C, at 1 bar of hydrogen and 50 to 55° C., in the presence of ethanol and water in a volume ratio of approximately 5 to 1. It is an example of a laboratory synthesis, working with a very dilute reaction mixture, not readily fit for industrial upscaling.

The selective N-alkylation of unprotected amino acids remains challenging. Most amino acids have limited solubility in nonpolar organic solvents, and their zwitterionic character renders these substrates sensitive to changes of pH and basic or acidic reagents.

Reductive amination of an organic feed substrate may occur in a suitable solvent. A properly chosen solvent may ensure solubility of the feed substrate and the amination product. Additionally, a solvent may bring advantages to such reductive amination reactions, such as an improved hydrogen solubility, a decreased viscosity of the reaction mixture, an improved mixing efficiency, an improved heat transfer, or a limited formation of undesirable by-products. Highly diluted reaction mixtures, however, result in poor space-time yields, which render the process economically unattractive. In case of highly concentrated reaction mixtures, the benefits of the solvent may be minimized, e.g., the reagents might not be sufficiently mixed. Also, there may be a tendency that either the organic feed substrates or reductive amination product thereof plug or “blind” the catalyst to hydrogen, slowing down the reductive amination. Such “blinding” of the catalyst will occur if one or more of the species present, whether the organic feed substrates or reductive amination product thereof, is sparingly soluble or otherwise strongly absorbed or adsorbed on the catalyst surface and thereby prevents approach of hydrogen molecules to the active catalytic sites.

There further remains a need for providing surfactants under environmentally beneficial conditions at high yield, in particular using bio-renewable starting materials, i.e., materials which are naturally available, which are available through biological processes, and/or which are available through chemical processes from naturally available materials. There is moreover a need for novel surfactants.

The present invention provides a process for the production of a surfactant of general formula (I)

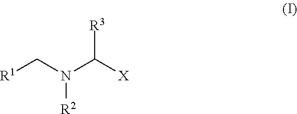

or a salt thereof, wherein
X is selected from COOH and CH₂SO₃H;
R¹ is selected from C₅-C₁₇-alkyl and C₅-C₁₇-alkenyl;
R² is selected from hydrogen, C₁-C₄-alkyl and CH₂PO₃H₂;
R³ is selected from hydrogen and C₁-C₈-alkyl, wherein C₁-C₈-alkyl is optionally substituted with one or more substituents selected from a hydroxy group, an amino group, a C₁-C₈-alkylamino group, a carboxyl group, a C₁-C₈-alkylcarboxylate group, a C₁-C₈-alkylamido group, a thio group, a C₁-C₈-alkylthio group, a guanidino group, and an aromatic group optionally substituted with a hydroxy group;
or wherein R² and R³, together with the nitrogen atom to which R² is bound and the carbon atom to which R³ is bound, form a 5- or 6-membered heterocycle, optionally substituted with a hydroxy group,
the process comprising reductive amination of an aldehyde of general formula (II)

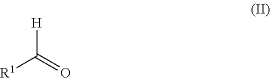

with an amino compound of general formula (III)

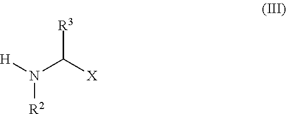

or a salt thereof, wherein R², R³ and X are defined as above;
in the presence of molecular hydrogen and a heterogeneous catalyst comprising a group 10 element of the periodic table of the elements;
wherein the reductive amination is conducted in feed operation, wherein the amino compound of general formula (III) is provided, and the aldehyde of general formula (II) is metered thereto; and
wherein the reductive amination is performed
   at a pressure of molecular hydrogen of at least 1 bara, preferably in the range of more than 1 bara to less than 50 bara;
   at a temperature of at least 25° C.; and
   in a solvent having a Hildebrand solubility parameter δ in the range of 18 to 38 MPa^{1/2}, at a solvent dilution ratio of less than 7.0 L per kg, the solvent dilution ratio being the total volume of solvent to the total weight of the aldehyde of general formula (II) and the amino compound of general formula (III).

The term solvent dilution ratio as used herein refers to the total volume of solvent to the total weight of the aldehyde of general formula (II) and the amino compound of general formula (III). The term “total volume of solvent” includes the initial volume of solvent as well as solvent volumes added over the course of the reductive amination. A solvent volume added over the course of the reductive amination is, for example, the solvent volume contained in a solution of a reactant that is metered in during a feed operation process. When the solvent is composed of a mixture of different solvents, the volume of the mixture may be smaller than the sum of the volumes of the individual solvents. This phenomenon is known as “volume contraction”. It may occur, e.g., when the solvent is composed of an aliphatic alcohol and water. It is understood that the “total volume of solvent” takes into account the volume contraction.

The process allows for high yields under mild reaction conditions and at limited reaction volumes. Moreover, the aldehydes of formula (II) and the amino compounds of formula (III) are typically naturally available, available through biological processes, and/or available through chemical processes from naturally available materials. Overall, the process thus allows for obtaining surfactants in an environmentally friendly fashion.

The process for the production of the surfactant of general formula (I) comprises reductive amination of an aldehyde of general formula (II) with an amino compound of general formula (III) or a salt thereof:

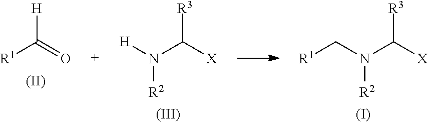

X is selected from COOH, CH₂SO₃H and COONa. Preferably, X is COOH

R¹ is selected from C₅-C₁₇-alkyl and C₅-C₁₇-alkenyl, and may be straight-chained or branched. When R¹ is an alkenyl group, R¹ may be monounsaturated or polyunsaturated. R¹ is preferably selected from C₇-C₁₅-alkyl and C₇-C₁₅-alkenyl, more preferably from C₇-C₁₃-alkyl and C₇-C₁₃-alkenyl, most preferably from C₇-C₁₁-alkyl and C₇-C₁₁-alkenyl.

In a preferred embodiment, R¹ is a C₈-, C₉- or C₁₁-alkyl, especially a C₈- or C₁₁-alkyl; or R¹ is a C₉-C₁₇-alkenyl, in particular C₉-alkenyl.

The aldehyde of general formula (II) is an aliphatic aldehyde. Suitable aldehydes of general formula (II) include octanal, nonanal, decanal, undecanal, dodecanal, tetradecanal, hexadecanal, 2-ethylhexanal and citral, in particular citral, dodecanal and tetradecanal. Citral is understood as a mixture of two geometric isomers, specifically geranial (citral A) and neral (citral B).

R² may be selected from hydrogen, C₁-C₄-alkyl, which may be straight-chained or branched, and CH₂PO₃H₂. Preferably, the C₁-C₄-alkyl is ethyl or methyl, in particular methyl.

R³ may be selected from hydrogen and optionally substituted C₁-C₈-alkyl, which may be straight-chained or branched. Preferably, the C₁-C₈-alkyl is optionally substituted C₁-C₆-alkyl or optionally substituted C₁-C₄-alkyl, in particular optionally substituted methyl, optionally substituted ethyl, optionally substituted n-propyl or optionally substituted n-butyl.

In a preferred embodiment, R² is hydrogen or methyl and R³ is optionally substituted C₁-C₄-alkyl.

R³ is optionally substituted with one or more substituents selected from a hydroxy group, an amino group, a C₁-C₈-alkylamino group, a carboxyl group, a C₁-C₈-alkylcarboxylate group, a C₁-C₈-alkylamido group, a thio group, a C₁-C₈-alkylthio group, a guanidino group, and an aromatic group, such as an indolyl group or imidazolyl group, wherein the aromatic group is optionally substituted with a hydroxy group.

In another embodiment, R² and R³, together with the nitrogen atom to which R² is bound and the carbon atom to which R³ is bound, form a 5- or 6-membered heterocycle, such as a pyrrolidinyl group or a piperidinyl group. The heterocycle may be saturated or unsaturated, preferably saturated. The heterocycle is optionally substituted with a hydroxy group.

Suitable amino compounds of general formula (III) include proteinogenic amino acids such as alanine, arginine, aspartic acid, asparagine, cysteine, selenocysteine, glycine, glutamic acid, glutamine, histidine, methionine, leucine, isoleucine, lysine, phenylalanine, proline, serine, threonine, tryptophan, tyrosine and valine, in particular proline; and non-proteinogenic amino acids, amino acid derivatives and amino acid analogues, such as glyphosate, hydroxyproline, pipecolic acid, pyrrolysine, ornithine, carnitine, β-N-methylamino-alanine, N-methyl-alanine, sarcosine, taurine and N-methyl taurine, in particular N-methyl taurine, N-methyl-alanine and sarcosine.

The surfactant may be a non-ionic compound of general formula (I), an internal (zwitterionic) salt, or an external salt of a compound of general formula (I). The amino compound of general formula (III) may be a non-ionic compound or an external salt of a compound of general formula (III). Even though the acid group of moiety X in formulae (I) and (III) is shown in its undissociated form, and the nitrogen atom carrying R² is shown in its unprotonated form, it is understood that the reaction product obtained in the present process may be an internal (zwitterionic) salt, wherein the acid group is dissociated and carries a negative charge, and the nitrogen atom is protonated and carries a positive charge. However, it is also possible that only one of said groups is charged and that this charge prevails, which is compensated by a counter-ion. These “external salts” may be obtained under acidic or basic conditions. The same is true for acidic and basic optional substituents.

In one embodiment, the process comprises neutralizing the surfactant of general formula (I) with an acid or a base, in particular an acid. Thus, an external salt of the surfactant of general formula (I) is obtained. In another embodiment, the amino compound of general formula (III) is an external salt. In this embodiment, an external salt of general formula (I) is directly obtained by reaction with the aldehyde of general formula (II).

The acid may be selected from organic acids and mineral acids. Suitable mineral acids include hydrochloric acid, sulfuric acid, amidosulfuric acid, and phosphoric acid. Preferably, the mineral acid is selected from hydrochloric acid and sulfuric acid.

Suitable organic acids include carboxylic acids, sulfonic acids, carbonic acid, organic phosphonic acids, and aminocarboxylic acids. Preferably, the organic acid is selected from carboxylic acids, most preferably from formic acid, acetic acid, oxalic acid, propionic acid, hydroxypropionic acid, lactic acid, malic acid, maleic acid, succinic acid, tartaric acid, aconitic acid, citric acid and glutamic acid.

The base may be selected from metal hydroxides and amines. Suitable metal hydroxides include alkali metals, in particular sodium hydroxide and potassium hydroxide. Sodium hydroxide is particularly preferred. Suitable amines include ammonia; aliphatic amines such as monomethylamine, dimethylamine, monoethylamine, diethylamine, monopropylamine, dipropylamine, decylamine, dodecylamine and tetradecylamine; alkanolamines such as ethanolamine and diethanolamine; and lysine. External salts obtained under basic conditions generally display improved solubility at higher pH values

Neutralization of the amine surfactant of general formula (I) is typically performed at room temperature, i.e., 25° C., and under atmospheric pressure. Neutralization is typically carried out in a solvent, preferably a solvent as described above. The neutralized reaction product may be purified by typical means, such as evaporation of the solvent to obtain a crude material, which may be further purified by, e.g., recrystallization.

The molar ratio of the total amount of the aldehyde of general formula (II) to the total amount of the amino compound of general formula (III) is preferably in the range of 0.8:1 to 1.3:1, more preferably 0.9:1 to 1.2:1, most preferably 0.95:1 to 1.15:1.

The reductive amination is performed in the presence of a heterogeneous catalyst comprising a group 10 element of the periodic table of the elements. The group 10 element may be selected from nickel (Ni), palladium (Pd) and platinum (Pt). Preferably, the group 10 element is selected from nickel (Ni) and palladium (Pd), in particular palladium (Pd).

The metals can be used as such, or else applied to supports. Preferred supports are selected from aluminum oxide, silicon dioxide, titanium dioxide, zirconium dioxide and activated carbon. Particular preference is given to the supported metals. Preferred supports are activated carbon, aluminum oxide and titanium dioxide. A very particularly preferred support is activated carbon. A very especially preferred hydrogenation catalyst is palladium on activated carbon.

In a preferred embodiment, the heterogeneous catalyst is selected from Raney nickel and palladium on activated carbon, in particular palladium on activated carbon (Pd/C). When the heterogeneous catalyst is palladium on activated carbon, the catalyst may comprise a binder such as polytetrafluoroethylene (PTFE).

The catalyst is typically present in an amount of at most 2.5 wt.-%, such as 0.01 to 2.5 wt.-%, preferably 0.01 to 1.5 wt.-%, more preferably 0.01 to 1.5 wt.-%, e.g., 0.3 to 1.2 wt.-% or 0.5 to 1.1 wt.-%, calculated as group 10 element relative to the weight of the amino compound of general formula (III). The inventive process thus requires relatively low amounts of catalyst.

The reductive amination is performed at a pressure of molecular hydrogen of at least 1 bara, preferably in the range of at least 1 bara to less than 50 bara, in particular more than 1 bara to less than 50 bara. Preferably, the pressure of molecular hydrogen is in the range of 3 to 30 bara, more preferably 3 to 25 bara, most preferably 4 to 21 bara. A pressure of molecular hydrogen of more than 1 bara allows for a favorably low reaction time, thus increasing the efficiency of the process.

On the other hand, if the pressure of molecular hydrogen is too high, the aldehyde is reduced to the corresponding alcohol, rather than partaking in the reductive amination.

The corresponding alcohol is notably difficult to separate from the reaction mixture. Thus, working at a pressure of molecular hydrogen within the claimed range allows for high selectivity and high efficiency of the process.

The reductive amination is performed at a temperature of at least 25° C. Preferably, the temperature is in the range of 25 to 120° C., more preferably 30 to 90° C., most preferably 40 to 50° C. A temperature in this range allows for the reductive amination reaction to proceed at an adequate rate under mild conditions.

The reductive amination is performed in the presence of a solvent having a Hildebrand solubility parameter δ in the range of 18 to 38 MPa^{1/2}, preferably in the range of 30 to 37 MPa^{1/2}, more preferably 31 to 37 MPa^{1/2}, most preferably 32 to 35 MPa^{1/2}. It was found that a solvent having a Hildebrand solubility parameter δ in this range allows for a high solubility of both the starting materials and the obtained surfactant. The solvent may be in the form of a single solvent or a mixture of two or more solvents. The Hildebrand solubility parameter δ provides a numerical estimate of the degree of interaction between compounds, and thus be used as an indicator of solubility. Compounds having similar values of 6 are likely miscible.

The Hildebrand solubility parameter δ is well-known in literature. The following table provides exemplary solvents and their Hildebrand solubility parameter from Barton, Allan F. M. (1983), Handbook of Solubility Parameters and Other Cohesion Parameters, CRC Press.

| solvent | δ [MPa^{1/2}] |
|---|---|
| n-pentane | 14.4 |
| n-hexane | 14.9 |
| n-heptane | 15.3 |
| diethyl ether | 15.4 |
| n-dodecane | 16.0 |
| cyclohexane | 16.8 |
| xylene | 18.2 |
| ethyl acetate | 18.2 |
| toluene | 18.3 |
| tetrahydrofuran | 18.5 |
| benzene | 18.7 |
| chloroform | 18.7 |
| methyl ethyl ketone | 19.3 |
| acetone | 19.7 |
| ethylene dichloride | 20.2 |
| methylene chloride | 20.2 |
| pyridine | 21.7 |
| morpholine | 22.1 |
| dimethylformamide | 24.7 |
| n-propanol | 24.9 |
| ethanol | 26.2 |
| dimethyl sulfoxide | 26.4 |
| n-butanol | 28.7 |
| methanol | 29.7 |
| propylene glycol | 30.7 |
| ethylene glycol | 34.9 |
| glycerol | 36.2 |
| water | 48.0 |

The Hildebrand solubility parameter δ of a solvent mixture may be determined by averaging the Hildebrand values of the individual solvents by volume. For example, the Hildebrand solubility parameter of a mixture of two parts ethanol and one part water may be calculated as:

$$δ = ( ( 2 \times 26.2 {MPa}^{1 / 2} + 48. {MPa}^{1 / 2} ) / 3 ) = 33.5 {MPa}^{1 / 2} .$$

Selection of a solvent having a Hildebrand solubility parameter δ in the above range ensures that the solvent has solubility characteristics suitable for the amino compound of general formula (III), and that the reductive amination reaction proceeds at an adequate rate.

In a preferred embodiment, the solvent comprises a protic solvent and/or an aprotic solvent comprising an ether moiety, in particular a protic solvent. A protic solvent is understood to be a solvent which is able to donate a proton (H⁺) to a solute, e.g., via an O—H or N—H bond, typically via hydrogen bonding. An aprotic solvent is unable to donate a proton to a solute.

Preferably, the solvent comprises at least one protic solvent selected from water and an aliphatic alcohol. Preferred aliphatic alcohols include methanol, ethanol and isopropanol, in particular ethanol. More preferably, the solvent comprises an aliphatic alcohol or a mixture of an aliphatic alcohol and water. In a preferred embodiment, the solvent is a mixture of an aliphatic alcohol and water in a volume ratio in the range of 1:1 to 4:1, preferably 1.25:1 to 3:1, most preferably 1.5:1 to 2:1. In a particularly preferred embodiment, the solvent is a mixture of ethanol and water in a volume ratio in the range of 1:1 to 4:1, preferably 1.25:1 to 4:1, more preferably 1.5:1 to 2:1, most preferably 1.5:1 to 2:1. The volume ratio is understood to relate to the ratio of the individual solvent volumes prior to mixing, i.e., disregarding any volume contraction which may occur upon mixing.

In another embodiment, the solvent comprises at least one aprotic solvent comprising an ether moiety, such as tetrahydrofuran, 2-methyltetrahydrofuran, 1,4-dioxane, or tetrahydropyran, in particular tetrahydrofuran. In one embodiment, the solvent is tetrahydrofuran.

In a further embodiment, the solvent comprises at least one aprotic solvent comprising an ether moiety and at least one protic solvent. For example, the solvent may comprise a mixture of tetrahydrofuran with at least one protic solvent, in particular water or an alcohol. In a preferred embodiment, the solvent is a mixture of tetrahydrofuran with water or ethanol, in particular a mixture of tetrahydrofuran with water.

The reductive amination is performed at a solvent dilution ratio of less than 7.0 L per kg, preferably at most 4.5 L per kg, more preferably at most 2.5 L per kg. Typically, the reductive amination is performed at a solvent dilution ratio of 1.0 to less than 7.0 L per kg, preferably 1.0 to 4.5 L per kg, more preferably 1.0 to 2.5 L per kg. A concentration of the aldehyde of general formula (II) and the amino compound of general formula (III) in this range allows for efficiently reacting the amino compound with the aldehyde at high selectivity. More diluted reaction mixtures result in poor space-time yields, while in some cases of too highly concentrated reaction mixtures, the benefits of the solvent may be minimized.

In one embodiment, the reductive amination is performed at a pressure of molecular hydrogen in the range of 3 to 30 bara, at a temperature in the range of 25 to 120° C., and in a solvent having a Hildebrand solubility parameter δ in the range of 18 to 38 MPa^{1/2}, at a solvent dilution ratio of 1.0 to 7.0 L per kg.

In a further embodiment, the reductive amination is performed at a pressure of molecular hydrogen in the range of 3 to 25 bara, preferably 4 to 21 bara, at a temperature in the range of 25 to 120° C., and in a solvent having a Hildebrand solubility parameter δ in the range of 18 to 38 MPa^{1/2}, at a solvent dilution ratio of 1.0 to 7.0 L per kg.

In a further embodiment, the reductive amination is performed at a pressure of molecular hydrogen in the range of 3 to 30 bara, at a temperature in the range of 30 to 90° C., more preferably 40 to 50° C., and in a solvent having a Hildebrand solubility parameter δ in the range of 18 to 38 MPa^{1/2}, at a solvent dilution ratio of 1.0 to 7.0 L per kg.

In a further embodiment, the reductive amination is performed at a pressure of molecular hydrogen in the range of 3 to 30 bara, at a temperature in the range of 25 to 120° C., and in a solvent having a Hildebrand solubility parameter δ in the range of 30 to 37 MPa^{1/2}, preferably 31 to 37 MPa^{1/2}, more preferably 32 to 35 MPa^{1/2}, at a solvent dilution ratio of 1.0 to 7.0 L per kg.

In a further embodiment, the reductive amination is performed at a pressure of molecular hydrogen in the range of 3 to 30 bara, at a temperature in the range of 25 to 120° C., and in a solvent having a Hildebrand solubility parameter δ in the range of 18 to 38 MPa^{1/2}, at a solvent dilution ratio of 1.0 to 4.5 L per kg, preferably 1.0 to 2.5 L per kg.

In a further embodiment, the reductive amination is performed at a pressure of molecular hydrogen in the range of 3 to 30 bara, at a temperature in the range of 25 to 120° C., and at a solvent dilution ratio of 1.0 to 7.0 L per kg, wherein the solvent is a mixture of an aliphatic alcohol, in particular ethanol, and water in a volume ratio of 1:1 to 4:1, preferably 1.25:1 to 3:1, most preferably 1.5:1 to 2:1.

In a further embodiment, the reductive amination is performed at a pressure of molecular hydrogen in the range of 3 to 30 bara, at a temperature in the range of 25 to 120° C., and in a solvent having a Hildebrand solubility parameter δ in the range of 18 to 38 MPam/z, at a solvent dilution ratio of 1.0 to 7.0 L per kg, wherein the heterogeneous catalyst is present in an amount of at most 2.5 wt.-%, such as 0.01 to 2.5 wt.-%, preferably 0.01 to 1.5 wt.-%, more preferably 0.01 to 1.5 wt.-%, e.g., 0.3 to 1.2 wt.-% or 0.5 to 1.2 wt.-%, calculated as the proportion of group 10 element relative to the total amount of the amino compound of general formula (III).

In a further embodiment, the reductive amination is performed at a pressure of molecular hydrogen in the range of 3 to 30 bara, at a temperature in the range of 25 to 120° C., and at a solvent dilution ratio of 1.0 to 7.0 L per kg, wherein the solvent is a mixture of an aliphatic alcohol and water, in particular ethanol and water, in a volume ratio of 1:1 to 4:1, preferably 1.25:1 to 3:1, most preferably 1.5:1 to 2:1, and wherein the heterogeneous catalyst is present in an amount of at most 2.5 wt.-%, such as 0.01 to 2.5 wt.-%, preferably 0.01 to 1.5 wt.-%, more preferably 0.01 to 1.5 wt.-%, e.g., 0.3 to 1.2 wt.-% or 0.5 to 1.2 wt.-%, calculated as the proportion of group 10 element relative to the total amount of the amino compound of general formula (III).

The process of the invention may be carried out in any reactor suitable for maintaining the pressure of molecular hydrogen in the desired range and at the desired temperature, such as an autoclave.

The process of the invention is conducted in feed operation, wherein the amino compound of general formula (III) is provided (i.e., initially charged), and the aldehyde of general formula (II) is metered thereto. It is understood that providing the amino compound of general formula (III) also includes providing a solution of the amino compound of general formula (III). Likewise, metering the aldehyde of general formula (II) thereto includes metering a solution of the aldehyde of general formula (II) thereto. The solvent contained in the solution of the amino compound of general formula (III) and the solvent contained in the solution of the aldehyde of general formula (II) together constitute the solvent in the presence of which the reductive amination occurs. The solvents comprised in the two solutions may be identical or different. In the latter case, the solvent in the presence of which the reductive amination occurs will be a mixture of solvents.

In general, the aldehyde of general formula (II) is added at constant feed rate. This allows for avoiding undesired side reactions. Thus, on the one hand, the reduction of the aldehyde to the corresponding alcohol is suppressed, obtaining the surfactant of general formula (I) at higher selectivity and avoiding the removal of the difficult to separate corresponding alcohol. On the other hand, aldol condensation of the aldehyde with itself to form the aldehyde dimer is avoided. This again allows for higher selectivity and moreover suppresses the formation of dimer-based surfactants with less advantageous properties.

The reaction product obtained in the process of the invention may be purified by typical means, such as filtration of the reaction mixture to remove the heterogeneous catalyst and subsequent evaporation of the solvent from the filtrate to obtain a crude material. In order to fully solubilize the reaction product in the obtained reaction mixture prior to filtration, a suitable additional solvent, in particular ethanol, may be added. The crude material may be further purified by, e.g., recrystallization.

In one embodiment, the aldehyde of general formula (II) and/or the amino compound of general formula (III) are bio-based compounds. In this embodiment, the process allows for obtaining surfactants in a particularly environmentally friendly fashion.

Using radiocarbon and isotope ratio mass spectrometry analysis, the bio-based content of materials can be determined. For example, ASTM International has established a standard method for assessing the bio-based content of materials. The ASTM method is designated ASTM-D6866.

The application of ASTM-D6866 to determine the “bio-based content” of materials is built on the same concepts as radiocarbon dating, but without use of the age equations. The analysis is performed by deriving a ratio of the amount of radiocarbon (¹⁴C) in an unknown sample to that of a modem reference standard. The ratio is reported as a percentage with the units “pMC” (percent modern carbon). If the material being analyzed is a mixture of present day radiocarbon and fossil carbon (containing no radiocarbon), then the pMC value obtained correlates directly to the amount of Biomass material present in the sample.

The modern reference standard used in radiocarbon dating is a NIST standard with a known radiocarbon content equivalent approximately to the year AD 1950. AD 1950 was chosen since it represented a time prior to thermo-nuclear weapons testing which introduced large amounts of excess radiocarbon into the atmosphere with each explosion (termed “bomb carbon”). The AD 1950 reference represents 100 pMC.

Further details for the assessment of materials with regard to determining whether or not they are bio-based may be found, e.g., in WO 2007/095262 A2.

The surfactants of general formula (I) are preferably at least partially biodegradable. Biodegradation is preferably at least 20%, more preferably at least 60% (all percentages in wt.-% based on the total solid content) within 28 days according to OECD 301.

It is believed that while the present process allows for avoiding a detrimentally high degree of aldol condensation of the aldehyde with itself to form the aldehyde dimer, and thus allows for higher selectivity, dimerization cannot be avoided altogether. It has now been found that a small amount of dimer-based surfactants has an advantageous effect on important surfactant properties, such as foam volume, wetting time, surface tension and contact angle.

The present invention thus further provides a surfactant composition, comprising a surfactant of general formula (I) or a salt thereof as defined above in an amount of at least 90 wt.-%, relative to the weight of the surfactant composition, and a compound of general formula (IV)

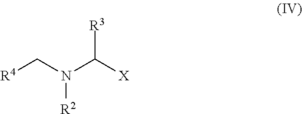

or a salt thereof in an amount of 0.01 to 5.0 wt.-%, relative to the weight of the surfactant composition,
wherein X, R² and R³ are defined as above; and R⁴ is selected from C₁₀-C₃₄-alkyl and C₁₀-C₃₄-alkenyl, with the proviso that the number of carbon atoms in R⁴ differs from the number of carbon atoms in R¹ of the surfactant of general formula (I), wherein the number of carbon atoms in R⁴ is preferably greater than the number of carbon atoms in R¹ in the surfactant of general formula (I), for example twice the number of carbon atoms in R¹ in the surfactant of general formula (I);
wherein the compound of general formula (IV) is preferably obtained via the reductive amination of an aldehyde dimer obtained from the self-aldol condensation of an aldehyde of formula (II) as defined above and an amino compound of general formula (III) as defined above.

In one embodiment, the surfactant composition comprises the compound of general formula (IV) in an amount of 0.01 to 4.5 wt.-%, preferably 0.05 to 4.5 wt.-%, more preferably 0.1 to 4.0 wt.-%, most preferably 0.5 to 3.0 wt.-%, relative to the weight of the surfactant composition.

In one embodiment, the surfactant composition comprises the surfactant of general formula (I) in an amount of at least 93 wt.-%, preferably at least 94 wt.-%, more preferably at least 95 wt.-%, such as at least 96 wt.-% or at least 97 wt.-%, relative to the weight of the surfactant composition.

The surfactant compositions may further comprise side products of the process of the invention, such as unreacted starting materials or side products, e.g., alcohols. Preferably, the surfactant composition comprises less than 5 wt.-%, more preferably less than 4 wt.-%, most preferably less than 3 wt.-%, of compounds besides the surfactant of general formula (I) and the compound of general formula (IV).

Preferably, the surfactant composition is obtained according to a process of the invention. The surfactant composition may be admixed with further components so as to obtain surfactants for a wide variety of applications, including detergents, as described in detail below.

In another embodiment, the present invention further provides a surfactant composition, comprising a surfactant of general formula (I) or a salt thereof as defined above and a compound of general formula (IV)

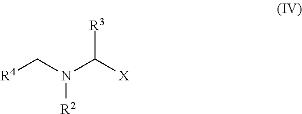

or a salt thereof,
wherein X, R² and R³ are defined as above; and R⁴ is selected from C₁₀-C₃₄-alkyl and C₁₀-C₃₄-alkenyl, with the proviso that the number of carbon atoms in R⁴ differs from the number of carbon atoms in R¹ of the surfactant of general formula (I), wherein the number of carbon atoms in R⁴ is preferably greater than the number of carbon atoms in R¹ in the surfactant of general formula (I), for example twice the number of carbon atoms in R¹ in the surfactant of general formula (I);
wherein the weight ratio of the total amount of surfactant of general formula (I) and salts thereof to the total amount of compound of general formula (IV) and salts thereof is in the range of 15:1 to 10,000:1, preferably 20:1 to 1,000:1, more preferably 25:1 to 100:1, such as 25:1 to 75:1;
wherein the compound of general formula (IV) is preferably obtained via the reductive amination of an aldehyde dimer obtained from the self-aldol condensation of an aldehyde of formula (II) as defined above and an amino compound of general formula (III) as defined above.

In the process of the invention, the surfactant of general formula (I) is obtained, together with a compound of general formula (IV) as a side product in small amounts. Preferably, the compound of general formula (IV) comprised in the surfactant compositions of the invention is derived from the self-aldol condensation product of the aldehyde (II) used in the process of the invention. The compound of general formula (IV) may be obtained in an enriched form from the crude reaction product of the process of the invention via, e.g., recrystallization. The proportions of the surfactant of general formula (I) and the compound of general formula (IV) may be determined via gas chromatography (after silylation) or via gas chromatography-mass spectrometry (GC-MS).

The invention moreover provides a compound selected from
(2S,4R)-N-n-nonyl-4-hydroxypyrrolidine-2-carboxylic acid,
(2S,4R)-1-(2-ethylhexyl)-4-hydroxypyrrolidine-2-carboxylic acid,
(2S,4R)-1-(3,7-dimethyloctyl)-4-hydroxypyrrolidine-2-carboxylic acid,
(3,7-dimethyloctyl)-L-proline,
N-n-nonyl-N-(phosphonomethyl)glycine,
N-(2-ethylhexyl)-N-(phosphonomethyl)glycine,
N-(3,7-dimethyloctyl)-N-(phosphonomethyl)glycine,
1-(3,7-dimethyloctyl)piperidine-2-carboxylic acid,
N-(3,7-dimethyloctyl)-N-methylglycine,
2-((2-ethylhexyl)(methyl)amino)ethane-1-sulfonic acid,
2-((3,7-dimethyloctyl)(methyl)amino)ethane-1-sulfonic acid,
N-(2-ethylhexyl)-N-methylalanine,
N-(3,7-dimethyloctyl)-N-methylalanine,
N-n-nonyl-L-leucine,
N-n-nonyl-L-isoleucine,
2-(nonylmethylamino)ethanesulfonic acid; and
N-n-nonyl-L-glutamic acid,
or a salt thereof.

The compounds of the invention may be obtained by the process of the invention.

The invention moreover provides a composition comprising at least one compound of the invention.

The invention further provides the use of a compound of the invention or of a composition of the invention as a surfactant. The surfactants and compositions of the invention, which are understood to include the surfactant compositions of the invention, can be used as surfactants in a wide variety of applications, including detergents such as granular laundry detergents, liquid laundry detergents, liquid dishwashing detergents; and in miscellaneous formulations such as all purpose cleaning agents, liquid soaps, shampoos, shower gels, and liquid scouring agents. Moreover, the compounds and compositions of the invention may be used as surfactants in institutional and industrial cleaning formulations such as kitchen cleaners, industrial laundry detergents, vehicle cleaners, and disinfection cleaners. Moreover, the compounds and compositions of the invention may be used as adjuvants in agrochemical formulations, such as pesticide formulations.

The compounds and compositions of the invention find particular use as surfactants in detergents, specifically laundry detergents and manual dishwashing products. These are generally comprised of a number of components besides the compound(s) of the invention. The composition typically comprises a total amount of 0.1 to 15 wt.-% of the compound(s) of the invention, relative to the weight of the composition. Typical compositions are known to the experts.

Laundry detergents typically comprise other surfactants of the anionic, nonionic, amphoteric or cationic type; builders such as phosphates, aminocarboxylates and zeolites; organic co-builders such as polycarboxylates; bleaching agents and their activators; foam controlling agents; enzymes; anti-greying agents; optical brighteners; and stabilizers.

Liquid laundry detergents generally comprise the same components as granular laundry detergents, but generally contain less of the builders. Moreover, liquid detergent formulations often comprise hydrotropic substances. All-purpose cleaning agents may comprise other surfactants, builders, foam suppressing agents, hydrotropes and solubilizer alcohols.

Builders may be comprised in amounts of up to 90% by weight, preferably about 5 to 35% by weight, to intensify the cleaning action. Examples of common inorganic builders are phosphates, polyphosphates, alkali metal carbonates, silicates and sulfates. Examples of organic builders are polycarboxylates, aminocarboxylates such as ethylenediaminetetraacetates, nitrilotriacetates, hydroxycarboxylates, citrates, succinates and substituted and unsubstituted alkanedi- and polycarboxylic acids.

Another type of builder, useful in granular laundry and built liquid laundry agents, includes various substantially water-insoluble materials which are capable of reducing the water hardness, e.g., by ion exchange processes. In particular, complex sodium aluminosilicates, known as type A zeolites, are useful for this purpose.

The laundry detergents may also contain bleaching agents, e.g., percompounds such as perborates, percarbonates, persulfates and organic peroxy acids. Formulations containing percompounds may also contain stabilizing agents, such as magnesium silicate, sodium ethylenediaminetetraacetate or sodium salts of phosphonic acids. In addition, bleach activators can be used to increase the efficiency of the inorganic persalts at lower washing temperatures. Particularly useful for this purpose are substituted carboxylic acid amides, e.g., tetraacetylethylenediamine, substituted carboxylic acids, e.g., isononyloxybenzenesulfonate and sodium cyanamide.

Examples of suitable hydrotropic substances are alkali metal salts of benzene, toluene and xylene sulfonic acids; alkali metal salts of formic acid, citric and succinic acid, urea, mono-, di-, and triethanolamine. Examples of solubilizer alcohols are ethanol, isopropanol, mono- or polyethylene glycols, monopropylene glycol and ether alcohols.

Examples of foam controlling agents are high molecular weight fatty acid soaps, paraffinic hydrocarbons, and silicon containing defoamers. In particular, hydrophobic silica particles having silicon adsorbed thereon are efficient foam control agents in these laundry detergent formulations.

Examples of known enzymes which are effective in laundry detergent agents are, among others, proteases, amylases, cellulases, mannanases, and lipases. Preference is given to enzymes which have their optimum performance at the design conditions of the detergent.

A large number of fluorescent whiteners are described in the literature. For laundry detergent formulations, the derivatives of diaminostilbene disulfonates and substituted distyrylbiphenyl are particularly suitable.

As anti-greying agents, water-soluble colloids of an organic nature are preferably used. Examples are water-soluble polyanionic polymers such as polymers and copolymers of acrylic and maleic acid, cellulose derivatives such as carboxymethyl cellulose, methyl- and hydroxyethylcellulose.

In addition to one or more of the aforementioned other surfactants and other detergent composition components, laundry detergent compositions typically comprise one or more inert components. For instance, the balance of liquid detergent composition is typically an inert solvent or diluent, most commonly water.

Manual dishwashing products may comprise, besides the compounds obtained in the process of the invention, other surfactants of the anionic, nonionic, amphoteric or cationic type; solvents; diamines; carboxylic acids or salts thereof; polymeric suds stabilizers; enzymes; builders; perfumes; and/or chelating agents.

Suitable solvents include diols, polymeric glycols, and mixtures thereof. Preferred diols include propylene glycol, 1,2 hexanediol, 2-ethyl-1,3-hexanediol and 2,2,4-trimethyl-1,3-pentanediol.

Polymeric glycols, which comprise ethylene oxide (EO) and propylene oxide (PO) groups, may also be included in the present invention. These materials are formed by adding blocks of ethylene oxide moieties to the ends of polypropylene glycol chains. A preferred polymeric glycol is a polypropylene glycol having an average molecular weight in the range of 1,000 to 5,000 g/mol. When polymeric glycols are present, it may be beneficial to include either a diol and/or an alkali metal inorganic salt, such as sodium chloride, so as to obtain satisfactory physical stability. Suitable amounts of diols to provide physical stability are in the amounts in the ranges found above.

Further suitable solvents include glycols or alkoxylated glycols, ethers and diethers having from 4 to 14 carbon atoms, preferably from 6 to 12 carbon atoms, aromatic alcohols, alkoxylated aromatic alcohols, aliphatic branched alcohols, alkoxylated aliphatic branched alcohols, linear C₁-C₈ alcohols, alkoxylated linear C₁-C₈ alcohols, C₈-C₁₄ alkyl and cycloalkyl hydrocarbons and halo hydrocarbons, C₆-C₁₆ glycol ethers, and mixtures thereof.

Suitable alkoxylated glycols are methoxy octadecanol and/or ethoxyethoxyethanol. Suitable aromatic alcohols include benzyl alcohol. Suitable aliphatic branched alcohols include 2-ethylbutanol and/or 2-methylbutanol. Suitable alkoxylated aliphatic branched alcohols include 1-methylpropoxyethanol and/or 2-methylbutoxyethanol. Suitable linear C₁-C₈ alcohols include methanol, ethanol, and/or propanol.

Manual dishwashing compositions typically comprise 0.01 to 20 wt.-% of solvent, based on the total weight of the composition. The solvents may be used in conjunction with an aqueous liquid carrier, such as water, or they may be used without any aqueous liquid carrier being present.

Manual dishwashing compositions may further comprise one or more diamines.

The composition preferably comprises 0.1 to 15 wt.-% of at least one diamine, based on the total weight of the composition.

Suitable diamines include organic diamines in which pK1 and pK2 are in the range of 8.0 to 11.5, such as 1,3-bis(methylamine)-cyclohexane (pKa=10 to 10.5), 1,3-propane diamine (pK1=10.5; pK2=8.8), 1,6-hexane diamine (pK1=11; pK2=10), 1,3-pentane diamine (pK1=10.5; pK2=8.9), 2-methyl-1,5-pentane diamine (Dytek A) (pK1=11.2; pK2=10.0). pKa values referenced herein may be obtained from literature, such as from “Critical Stability Constants: Volume 2, Amines” by Smith and Martel, Plenum Press, NY and London, 1975. The pKa of the diamines is specified in an all-aqueous solution at 25° C. and for an ionic strength between 0.1 to 0.5 M.

Other preferred materials are primary diamines having two primary amino groups with alkylene spacers ranging from C₄ to C₈.

The compositions according to the present invention may comprise a linear or cyclic carboxylic acid or salt thereof. Where the acid or salt thereof is present and is linear, it preferably comprises from 1 to 6 carbon atoms whereas where the acid is cyclic, it preferably comprises greater than 3 carbon atoms. The linear or cyclic carbon-containing chain of the carboxylic acid or salt thereof may be substituted with a substituent group selected from the group consisting of hydroxyl, ester, ether, aliphatic groups having from 1 to 6, more preferably 1 to 4 carbon atoms and mixtures thereof.

Preferred carboxylic acids are those selected from the group consisting of salicylic acid, maleic acid, acetyl salicylic acid, 3-methyl salicylic acid, 4-hydroxy isophthalic acid, dihydroxyfumaric acid, 1,2,4-benzene tricarboxylic acid, pentanoic acid and salts thereof and mixtures thereof. Where the carboxylic acid exists in the salt form, the cation of the salt is preferably selected from alkali metal, alkaline earth metal, monoethanolamine, diethanolamine or triethanolamine and mixtures thereof.

The carboxylic acid or salt thereof is preferably present in an amount from 0.1% to 5 wt.-%, based on the total weight of the composition.

The composition may comprise a polymeric suds stabilizer. These polymeric suds stabilizers provide extended suds volume and suds duration without sacrificing the grease cutting ability of the liquid detergent compositions. Suitable polymeric suds stabilizers include homopolymers of (N,N-di(C₁-C₈ alkyl)amino)(C₁-C₈)alkyl acrylate esters; and copolymers thereof.

The molecular weight of the polymeric suds stabilizers is preferably in the range of 1,000 to 2,000,000 g/mol, most preferably from 20,000 to 500,000 g/mol. Polymeric suds stabilizer may be present in the form of a salt, for example the citrate, sulfate, or nitrate salt of (N,N-dimethylamino)alkyl acrylate ester. One preferred polymeric suds stabilizer is (N,N-dimethylamino)alkyl acrylate ester. Polymeric suds stabilizers are preferably present in an amount of 0.01% to 15 wt.-%, based on the total weight of the composition.

Suitable builders include aluminosilicate materials, silicates, polycarboxylates and fatty acids, materials such as ethylene-diamine tetraacetate, metal ion sequestrants such as aminopolyphosphonates, particularly ethylenediamine tetramethylene phosphonic acid and diethylene triamine pentamethylene-phosphonic acid. Though less preferred for obvious environmental reasons, phosphate builders can also be used.

Suitable polycarboxylate builders include citric acid, preferably in the form of a water-soluble salt, and derivatives of succinic acid. Specific examples include lauryl succinate, myristyl succinate, palmityl succinate 2-dodecenylsuccinate, 2-tetradecenyl succinate.

Succinate builders are preferably used in the form of their water-soluble salts, including sodium, potassium, ammonium and alkanolammonium salts. Other suitable polycarboxylates are oxodisuccinates and mixtures of tartrate monosuccinic and tartrate disuccinic acid, as described in U.S. Pat. No. 4,663,071.

Suitable fatty acid builders include saturated and unsaturated C₁₀₋₁₈ fatty acids, as well as the corresponding soaps. Preferred saturated species have from 12 to 16 carbon atoms in the alkyl chain. The preferred unsaturated fatty acid is oleic acid. Other preferred builder system for liquid compositions is based on dodecenyl succinic acid and citric acid.

Builders are preferably present in amounts of 0.5% to 50 wt.-%, more preferably 5 to 25 wt.-%, based on the total weight of the composition.

Suitable enzymes include enzymes selected from cellulases, hemicellulases, peroxidases, proteases, gluco-amylases, amylases, lipases, cutinases, pectinases, xylanases, reductases, oxidases, phenoloxidases, lipoxygenases, ligninases, pullulanases, tannases, pentosanases, malanases, β-glucanases, arabinosidases or mixtures thereof. Enzymes may be present in amounts of 0.0001% to 5 wt.-% of active enzyme, based on the total weight of the composition.

Preferred proteolytic enzymes, then, are selected from the group consisting of Alcalase® (Novo Industri A/S), BPN′, Protease A and Protease B (Genencor), and mixtures thereof. Protease B is most preferred. Preferred amylase enzymes include TERMAMYL®, DURAMYL® and the amylase enzymes those described in WO 9418314 to Genencor International and WO 9402597 to Novo.

Suitable chelating agents include iron and/or manganese chelating agents. Such chelating agents can be selected from the group consisting of amino carboxylates, amino phosphonates, polyfunctionally-substituted aromatic chelating agents and mixtures thereof. Suitable amino carboxylates include ethylenediaminetetraacetates, N-hydroxyethylethylenediaminetriacetates, nitrilo-tri-acetates, ethylenediamine tetraproprionates, triethylenetetraaminehexaacetates, diethylenetriaminepentaacetates, and ethanoldiglycines, alkali metal, ammonium, and substituted ammonium salts thereof, and mixtures thereof. Suitable amino phosphonates include ethylenediaminetetrakis (methylenephosphonates). Suitable polyfunctionally-substituted aromatic chelating agents include dihydroxydisulfobenzenes such as 1,2-dihydroxy-3,5-disulfobenzene.

Chelating agents may be present in amounts of 0.00015% to 15 wt.-%, based on the weight of the composition.

The invention is described in further detail by the subsequent examples.

### EXAMPLES

### General Information

Reductive aminations of an aldehyde with an amino compound were performed at a temperature of 25 to 90° C. and at a hydrogen pressure of 5 to 20 bar in a 300 mL autoclave in accordance with Table 1 below.

Analysis was performed via gas chromatography (after silylation) or via gas chromatography-mass spectrometry (GC-MS), and structures were confirmed by nuclear magnetic resonance (NMR) spectroscopy.

### Methods

The foam volume was measured according to EN 12728 at 2 g/L in tap water with a hardness of 10° d.

The cotton wetting time was measured according to EN 1772 at 1 g/L in deionized water with 10 wt.-% of Na₂CO₃.

The dynamic surface tension was measured at 23° C. after 0.10 s, at 1 g/L in deionized water with a SITA T60online bubble pressure tensiometer.

The interfacial tension vs. hexadecane was measured at 23° C. after 10 min, at 1 g/L in deionized water with a DataPhysics instrument OCA25.

The contact angle was measured on polyethylene at 40° C. after 10.0 s, at 0.2 g/L in deionized water with a DataPhysics instrument OCA25.

### Comparative Example 1

Palladium on carbon black (10 wt.-%, 2.5 g) was added to a solution of L-proline (99%, 25 g) and dodecanal (95%, 43 g) in tetrahydrofuran (120 mL) in an autoclave. The reaction vessel was closed and subsequently purged with nitrogen gas (thrice at 5 bar) and hydrogen gas (thrice at 5 bar). At an initial hydrogen gas pressure of 5 bar, stirring (700 rpm) was applied. The reaction mixture was warmed to 30° C. and the hydrogen pressure was maintained at 5 bar.

The reaction mixture was stirred under these conditions for 12 h, then cooled to room temperature and purged with nitrogen gas (thrice at 5 bar). Afterwards, the catalyst was filtered off and the filter cake was washed thrice with tetrahydrofuran. The solvent was removed by evaporation, yielding the crude material as a grey solid (purity: 84%, 61 g, yield: 83%). Recrystallization from heptane/toluene (19:1) yielded the product as a mica-like solid (36 g, purity: 90%, yield: 53%).

### Comparative Examples 2 to 7

Comparative Examples 2 to 7 were performed analogously to Comparative Example 1, as indicated in Table 1.

### Comparative Example 8

Palladium on carbon black (10 wt.-%, 1 g) was added to a solution of trans-4-hydroxy-L-proline (99%, 10 g) and dodecanal (95%, 16 g) in a mixture of tetrahydrofuran (50 mL) and water (50 mL) in an autoclave. The reaction vessel was closed and subsequently purged with nitrogen gas (thrice at 5 bar) and hydrogen gas (thrice at 5 bar). At an initial hydrogen pressure of 20 bar, stirring (700 rpm) was applied. The reaction mixture was warmed to 90° C. and the hydrogen pressure was maintained at 20 bar.

The reaction mixture was stirred under these conditions for 24 h, then cooled to room temperature and purged with nitrogen gas (thrice at 5 bar). Afterwards, the catalyst was filtered off and the filter cake was washed thrice with tetrahydrofuran. The solvent was removed by evaporation, yielding the crude material as a solid (15 g, purity: 81%, yield: 52%).

### Comparative Example 9

Palladium on carbon black (10 wt.-%, 2 g) was added to a solution of L-proline (99%, 20 g) and dodecanal (95%, 35 g) in ethanol (135 mL) in an autoclave. The reaction vessel was closed and subsequently purged with nitrogen gas (thrice at 5 bar) and hydrogen gas (thrice at 5 bar). At an initial hydrogen pressure of 5 bar, stirring (700 rpm) was applied. The reaction mixture was warmed to 25° C. and the hydrogen pressure was maintained at 5 bar.

The reaction mixture was stirred under these conditions for 12 h, then cooled to room temperature and purged with nitrogen gas (thrice at 5 bar). Afterwards, the catalyst was filtered off (the filter cake was washed twice with ethanol) and the solvent was removed by evaporation, yielding the crude material as a solid (53 g, purity: 72%, yield: 79%).

### Comparative Example 10

Comparative Example 10 was performed analogously to Comparative Example 9, as indicated in Table 1.

### Comparative Example 11

Palladium on carbon black (10 wt.-%, 2 g) was added to a solution of sarcosine (100%, 20 g) in water (100 mL) in an autoclave. The reaction vessel was closed and subsequently purged with nitrogen gas (thrice at 5 bar) and hydrogen gas (thrice at 5 bar). At an initial hydrogen pressure of 20 bar, stirring (700 rpm) was applied. The reaction mixture was warmed to 50° C. Subsequently, dodecanal (95%, 46 g) was metered continuously to the reaction mixture over the course of 6 h. The temperature (50° C.) and hydrogen pressure (20 bar) were maintained.

After complete addition of the aldehyde, the reaction mixture was stirred at a temperature of 50° C. and a hydrogen pressure of 20 bar for 6 h, then cooled to room temperature and purged with nitrogen gas (thrice at 5 bar). Afterwards, the catalyst was filtered off (the filter cake was washed with ethanol) and the solvent was removed by evaporation, yielding the crude material as a solid (61 g, purity: 49%, yield: 52%).

### Example 12

Palladium on carbon black (10 wt.-%, 2 g) was added to a solution of L-proline (100%, 20 g) in a mixture of ethanol (50 mL) and water (40 mL) in an autoclave. The reaction vessel was closed and subsequently purged with nitrogen gas (thrice at 5 bar) and hydrogen gas (thrice at 5 bar). At an initial hydrogen pressure of 5 bar, stirring (700 rpm) was applied. The reaction mixture was warmed to 40° C. Subsequently, dodecanal (95%, 35 g) as a solution in ethanol (20 mL) was metered continuously to the reaction mixture over the course of 5 h. The temperature (40° C.) and hydrogen pressure (5 bar) were maintained.

After complete addition of the aldehyde solution, the reaction mixture was stirred at a temperature of 40° C. and a hydrogen pressure of 5 bar for 1 h, then cooled to room temperature and purged with nitrogen gas (thrice at 5 bar). Afterwards, the catalyst was filtered off and the filter cake was washed twice with ethanol. The solvent was removed by evaporation, yielding the crude material as a solid (51 g, purity: 92%, yield: 96%).

### Examples 13 to 21

Examples 13 to 21 were performed analogously to Example 9, as indicated in Table 1.

### Comparative Example 22

Palladium on carbon black (3 wt.-%, 5.2 g) was added to a solution of rac-pipecolic acid (99%, 5.2 g) and dodecanal (95%, 9.7 g) in a mixture of ethanol (102 mL) and water (20.5 mL) in an autoclave. The reaction vessel was closed and subsequently purged with nitrogen gas (thrice at 5 bar) and hydrogen gas (thrice at 5 bar). At an initial hydrogen gas pressure of 1 bar, stirring (700 rpm) was applied. The reaction mixture was warmed to 55° C. and the hydrogen pressure was maintained at 1 bar.

The reaction mixture was stirred under these conditions for 4 h, then cooled to room temperature and purged with nitrogen gas (thrice at 5 bar). Afterwards, the catalyst was filtered off (the filter cake was washed thrice with ethanol and water each) and the solvent was removed by evaporation, yielding the crude material as a solid (14 g, purity: 70%, yield: 79%).

### Examples 23 to 33

Examples 23 to 33 were performed analogously to Example 12, as indicated in Table 1.

TABLE 1

| | | Solvent | | | | | | | | | |
|---|---|---|---|---|---|---|---|---|---|---|---|
| | | Dilution | δ ² | | Amino | | Time | H₂ | T | Yield | Purity |
| # | Solvent | Ratio ¹ | [MPa^{1/2}] | Catalyst | Compound | Aldehyde | [h] | [bara] | [° C.] | [%] | [%] |
| 1 * | THF | 1.8 | 18.5 | Pd/C ³ | L-proline | dodecanal | 12 | 5 | 30 | 83 | 84 |
| | (125 mL) | | | (2.5 g) | (25 g) | (43 g) | | | | | |
| 2 * | THF | 4.3 | 18.5 | Pd/C ³ | L-proline | nonanal | 24 | 5 | 30 | 82 | 85 |
| | (100 mL) | | | (1.0 g) | (10 g) | (13 g) | | | | | |
| 3 * | THF | 1.8 | 18.5 | Pd/C ³ | sarcosine | dodecanal | 24 | 20 | 50 | 91 | 90 |
| | (120 mL) | | | (2.0 g) | (20 g) | (46 g) | | | | | |
| 4 * | THF | 3.7 | 18.5 | Pd/C ³ | sarcosine | nonanal | 24 | 20 | 50 | 75 ⁴ | 97 ⁴ |
| | (100 mL) | | | (1.0 g) | (10 g) | (17 g) | | | | | |
| 5 * | THF | 4.6 | 18.5 | Pd/C ³ | rac- | nonanal | 24 | 5 | 30 | 72 | 89 |
| | (100 mL) | | | (1.0 g) | pipecolic | (12 g) | | | | | |
| | | | | | acid | | | | | | |
| | | | | | (10 g) | | | | | | |
| 6 * | THF | 4.5 | 18.5 | Pd/C ³ | rac- | dodecanal | 24 | 5 | 30 | 91 ⁴ | 95 ⁴ |
| | (120 mL) | | | (1.0 g) | pipecolic | (17 g) | | | | | |
| | | | | | acid | | | | | | |
| | | | | | (10 g) | | | | | | |
| 7 * | THF | 6.9 | 18.5 | Pd/C ³ | N-methyl | nonanal | 24 | 20 | 50 | 40 ⁴ | 96 ⁴ |
| | (150 mL) | | | (1.0 g) | taurine | (12 g) | | | | | |
| | | | | | (10 g) | | | | | | |
| 8 * | THF/H₂O | 3.9 | 33.3 | Pd/C ³ | trans-4- | dodecanal | 24 | 20 | 90 | 52 | 81 |
| | 1:1 | | | (1.0 g) | hydroxy-L- | (16 g) | | | | | |
| | (100 mL) | | | | proline | | | | | | |
| | | | | | (10 g) | | | | | | |
| 9 * | EtOH | 2.4 | 26.2 | Pd/C ³ | L-proline | dodecanal | 12 | 5 | 25 | 79 | 72 |
| | (135 mL) | | | (2.0 g) | (20 g) | (35 g) | | | | | |
| 10 * | EtOH | 1.8 | 26.2 | Pd/C ³ | sarcosine | dodecanal | 24 | 20 | 50 | 78 | 86 |
| | (110 mL) | | | (2.0 g) | (20 g) | (46 g) | | | | | |
| 11 * | H₂O | 1.5 | 48.0 | Pd/C ³ | sarcosine | dodecanal | 6 + 6 ⁵ | 20 | 50 | 52 | 49 |
| | (100 mL) | | | (2.0 g) | (20 g) | (46 g) | | | | | |
| 12 | EtOH/H₂O | 1.9 | 34.1 | Pd/C ³ | L-proline | dodecanal | 5 + 1 ⁵ | 5 | 40 | 96 | 92 |
| | 1.75:1 | | | (2.0 g) | (20 g) | (35 g) | | | | | |
| | (110 mL) | | | | | | | | | | |
| 13 | EtOH/H₂O | 1.8 | 34.1 | Pd/C ³ | L-proline | citral ⁷ | 5 + 1 ⁵ | 5 | 40 | 64 | 71 |
| | 1.75:1 | | | (2.0 g) | (20 g) | (29 g) | | | | | |
| | (110 mL) | | | | | | | | | | |
| 14 | EtOH/H₂O | 2.3 | 34.1 | Pd/C ³ | L-proline | 2-ethyl- | 5 + 1 ⁵ | 5 | 40 | 82 | 94 |
| | 1.75:1 | | | (2.0 g) | (20 g) | hexanal | | | | | |
| | (110 mL) | | | | | (26 g) | | | | | |
| 15 | EtOH/H₂O | 1.6 | 34.1 | Pd/C ³ | sarcosine | dodecanal | 5 + 1 ⁵ | 20 | 50 | 93 | 93 |
| | 1.75:1 | | | (2.0 g) | (20 g) | (48 g) | | | | | |
| | (110 mL) | | | | | | | | | | |
| 16 | EtOH/H₂O | 1.8 | 34.1 | Pd/C ³ | sodium | dodecanal | 5 + 1 ⁵ | 20 | 50 | 77 | 82 |
| | 1.75:1 | | | (2.0 g) | sarcosinate | (38 g) | | | | | |
| | (110 mL) | | | | (20 g) | | | | | | |
| 17 | EtOH/H₂O | 1.9 | 34.1 | Pd/C ³ | sarcosine | citral ⁷ | 5 + 1 ⁵ | 20 | 50 | 82 | 87 |
| | 1.75:1 | | | (2.0 g) | (20 g) | (38 g) | | | | | |
| | (110 mL) | | | | | | | | | | |
| 18 | EtOH/H₂O | 2.0 | 34.1 | Pd/C ³ | sarcosine | 2-ethyl- | 5 + 1 ⁵ | 20 | 50 | 90 | 93 |
| | 1.75:1 | | | (2.0 g) | (20 g) | hexanal | | | | | |
| | (110 mL) | | | | | (33 g) | | | | | |
| 19 | EtOH/H₂O | 1.9 | 34.1 | Pd/C ³ | DL-proline | dodecanal | 5 + 1 ⁵ | 5 | 50 | 90 | 94 |
| | 1.75:1 | | | (2.0 g) | (20 g) | (37 g) | | | | | |
| | (110 mL) | | | | | | | | | | |
| 20 | EtOH/H₂O | 1.7 | 34.1 | Pd/C ³ | N-methyl-L- | dodecanal | 5 + 1 ⁵ | 5 | 40 | 72 | 71 |
| | 1.75:1 | | | (2.0 g) | alanine | (41 g) | | | | | |
| | (110 mL) | | | | (20 g) | | | | | | |
| 21 | EtOH/H₂O | 2.0 | 34.1 | Pd/C ³ | rac- | dodecanal | 5 + 1 ⁵ | 5 | 40 | 87 | 80 |
| | 1.75:1 | | | (2.0 g) | pipecolic | (33 g) | | | | | |
| | (110 mL) | | | | acid | | | | | | |
| | | | | | (20 g) | | | | | | |
| 22 * | EtOH/H₂O | 8.2 | 29.8 | Pd/C ⁶ | rac- | dodecanal | 4 | 1 | 55 | 79 | 70 |
| | 5:1 | | | (5.2 g) | pipecolic | (9.7 g) | | | | | |
| | (122.5 mL) | | | | acid | | | | | | |
| | | | | | (5.2 g) | | | | | | |
| 23 * | EtOH/H₂O | 16.2 | 34.1 | Pt/C ⁸ | sarcosine | dodecanal | 5 + 1 ⁵ | 5 | 40 | 63 | 64 |
| | 1.75:1 | | | (0.8 g) | (2.0 g) | (4.6 g) | | | | | |
| | (110 mL) | | | | | | | | | | |
| 24 | EtOH/H₂O | 1.6 | 34.1 | Pd/C ³ | sarcosine | dodecanal | 5 + 1 ⁵ | 5 | 10 | 76 | 76 |
| | 1.75:1 | | | (2.0 g) | (20) | (46) | | | | | |
| | (110 mL) | | | | | | | | | | |
| 25 | EtOH/H₂O | 3.2 | 34.1 | Pt/C ⁸ | sarcosine | dodecanal | 5 + 1 ⁵ | 20 | 50 | 82 | 95 |
| | 1.75:1 | | | (4.0 g) | (10 g) | (23 g) | | | | | |
| | (110 mL) | | | | | | | | | | |
| 26 | EtOH/H₂O | 3.8 | 34.1 | Pt/C ⁸ | L-proline | dodecanal | 5 + 1 ⁵ | 20 | 50 | 86 | 87 |
| | 1.75:1 | | | (4.0 g) | (10 g) | (18 g) | | | | | |
| | (110 mL) | | | | | | | | | | |
| 27 * | EtOH/H₂O | 1.5 | 34.1 | Pd/C ³ | L-proline | Dodecanal | 5 + 1 ⁵ | 5 | 10 | 0 | 0 |
| | 1.75:1 | | | (2.0 g) | (26 g) | (46) | | | | | |
| | (110 mL) | | | | | | | | | | |
| 28a | EtOH/H₂O | 2.7 | 34.1 | Pd/C ⁹ | L-proline | dodecanal | 18 + 2 ⁵ | 5 | 40 | 95 | 94 |
| | 1.75:1 | | | (9.0 g) | (90 g) | (159 g) | | | | | |
| | (660 mL) | | | | | | | | | | |
| 28b | EtOH/H₂O | 2.7 | 34.1 | Pd/C ^{9, 10} | L-proline | dodecanal | 18 + 2 ⁵ | 5 | 40 | 67 ¹¹ | 72 ¹¹ |
| | 1.75:1 | | | (9.0 g) | (90 g) | (159 g) | | | | | |
| | (660 mL) | | | | | | | | | | |
| 28c | EtOH/H₂O | 2.7 | 34.1 | Pd/C ^{9, 10} | L-proline | dodecanal | 18 + 2 ⁵ | 5 | 40 | 32 ¹¹ | 39 ¹¹ |
| | 1.75:1 | | | (9.0 g) | (90 g) | (159 g) | | | | | |
| | (660 mL) | | | | | | | | | | |
| 29a | EtOH/H₂O | 2.7 | 34.1 | Pd/C ⁹ | L-proline | dodecanal | 18 + 2 ⁵ | 20 | 50 | 76 | 90 |
| | 1.75:1 | | | (9.0 g) | (90 g) | (159 g) | | | | | |
| | (660 mL) | | | | | | | | | | |
| 29b | EtOH/H₂O | 2.7 | 34.1 | Pd/C ^{9, 10} | L-proline | dodecanal | 18 + 2 ⁵ | 20 | 50 | 88 | 94 |
| | 1.75:1 | | | (9.0 g) | (90 g) | (159 g) | | | | | |
| | (660 mL) | | | | | | | | | | |
| 29c | EtOH/H₂O | 2.7 | 34.1 | Pd/C ^{9, 10} | L-proline | dodecanal | 18 + 2 ⁵ | 20 | 50 | 99 | 96 |
| | 1.75:1 | | | (9.0 g) | (90 g) | (159 g) | | | | | |
| | (660 mL) | | | | | | | | | | |
| 30 ¹² | EtOH/H₂O | 2.4 | 35.1 | Pd/C ⁹ | L-proline | dodecanal | 8 + 1 ⁵ | 5 | 50 | 85 | 86 |
| | 1.46:1 | | | (9.0 g) | (90 g) | (159 g) | | | | | |
| | (590 mL) | | | | | | | | | | |
| 31a ¹² | EtOH/H₂O | 2.0 | 34.1 | Pd/C ⁹ | L-proline | dodecanal | 18 + 2 ⁵ | 20 | 50 | 80 | 94 |
| | 1.75:1 | | | (9.0 g) | (114 g) | (202 g) | | | | | |
| | (660 mL) | | | | | | | | | | |
| 31b ¹² | EtOH/H₂O | 2.0 | 34.1 | Pd/C ^{9, 10} | L-proline | dodecanal | 18 + 2 ⁵ | 20 | 50 | 83 | 94 |
| | 1.75:1 | | | (9.0 g) | (114 g) | (202 g) | | | | | |
| | (660 mL) | | | | | | | | | | |
| 31c ¹² | EtOH/H₂O | 2.0 | 34.1 | Pd/C ^{9, 10} | L-proline | dodecanal | 18 + 2 ⁵ | 20 | 50 | 94 | 96 |
| (196) | 1.75:1 | | | (9.0 g) | (114 g) | (202 g) | | | | | |
| | (660 mL) | | | | | | | | | | |
| 32a ¹² | EtOH/H₂O | 2.2 | 34.1 | Pd/C ⁹ | sarcosine | dodecanal | 18 + 2 ⁵ | 20 | 50 | 99 | 98 |
| | 1.75:1 | | | (9.0 g) | (90 g) | (202 g) | | | | | |
| | (660 mL) | | | | | | | | | | |
| 32b ¹² | EtOH/H₂O | 2.2 | 34.1 | Pd/C ^{9, 10} | sarcosine | dodecanal | 18 + 2 ⁵ | 20 | 50 | 97 | 98 |
| | 1.75:1 | | | (9.0 g) | (90 g) | (202 g) | | | | | |
| | (660 mL) | | | | | | | | | | |
| 32c ¹² | EtOH/H₂O | 2.2 | 34. | Pd/C ^{9, 10} | sarcosine | dodecanal | 18 + 2 ⁵ | 20 | 50 | 97 | 98 |
| | 1.75:1 | | | (9.0 g) | (90 g) | (202 g) | | | | | |
| | (660 mL) | | | | | | | | | | |
| 33a | EtOH/H₂O | 2.0 | 34.1 | Pt/C ⁸ | L-proline | dodecanal | 5 + 1 ⁵ | 20 | 50 | 93 | 94 |
| | 1.75:1 | | | (9.0 g) | (57 g) | (101 g) | | | | | |
| | (330 mL) | | | | | | | | | | |
| 33b ¹³ | EtOH/H₂O | 2.0 | 34.1 | Pt/C ^{8, 10} | L-proline | dodecanal | 5 + 1 ⁵ | 20 | 50 | 90 | 96 |
| | 1.75:1 | | | (9.0 g) | (57 g) | (101 g) | | | | | |
| | (330 ml) | | | | | | | | | | |

¹ volume contractions were taken into account for mixtures of two solvents
² Hildebrandt solubility parameter
³ 10 wt.-% palladium on carbon black (no water content)
⁴ after recrystallization
⁵ time for continuous dosing of the aldehyde, plus additional stirring time
⁶ 3,7-dimethylocta-2,6-dienal (mixture of isomers)
⁷ 3 wt.-% palladium on carbon black (no water content)
⁸ 5 wt.-% platinum on carbon black (50 wt.-% water content)
⁹ 5 wt.-% palladium on carbon black (50 wt.-% water content)
¹⁰ catalyst recycled after previous run
¹¹ decreasing values observed due to catalyst poisoning (formation of oxazolidinone)
¹² stirring at 850 rpm
¹³ stirring at 500 rpm
* comparative example

### Example 34

Palladium on carbon black (10 wt.-%, 2 g) was added to a mixture of L-proline (98%, 20 g) in ethanol (70 mL) in an autoclave. The reaction vessel was closed and subsequently purged with nitrogen gas (thrice at 5 bar) and hydrogen gas (thrice at 5 bar). At an initial hydrogen pressure of 5 bar, stirring (700 rpm) was applied. The reaction mixture was warmed to 30° C. Subsequently, dodecanal (95%, 66 g) as a solution in ethanol (20 mL) was metered continuously to the reaction mixture over the course of 6 h. The temperature (30° C.) and hydrogen pressure (5 bar) were maintained.

After complete addition of the aldehyde solution, the reaction mixture was stirred at a temperature of 30° C. and a hydrogen pressure of 5 bar for 6 h, then cooled to room temperature and purged with nitrogen gas (thrice at 5 bar). Afterwards, the catalyst was filtered off and the filter cake was washed twice with ethanol. The solvent was removed by evaporation, yielding the crude material as a solid.

### Example 35

Palladium on carbon black (10 wt.-%, 2 g) was added to a mixture of L-proline (98%, 20 g) and dodecanal (95%, 66 g) in ethanol (90 mL) in an autoclave. The reaction vessel was closed and subsequently purged with nitrogen gas (thrice at 5 bar) and hydrogen gas (thrice at 5 bar). At an initial hydrogen pressure of 5 bar, stirring (700 rpm) was applied. The reaction mixture was warmed to 30° C. The temperature (30° C.) and hydrogen pressure (5 bar) were maintained and the reaction mixture was stirred 12 h, then cooled to room temperature and purged with nitrogen gas (thrice at 5 bar). Afterwards, the catalyst was filtered off and the filter cake was washed twice with ethanol. The solvent was removed by evaporation, yielding the crude material as a solid.

### Example 36

Palladium on carbon black (10 wt.-%, 2 g) was added to a mixture of sarcosine (100%, 20 g) in ethanol (50 mL) in an autoclave. The reaction vessel was closed and subsequently purged with nitrogen gas (thrice at 5 bar) and hydrogen gas (thrice at 5 bar). At an initial hydrogen pressure of 5 bar, stirring (700 rpm) was applied. The reaction mixture was warmed to 30° C. Subsequently, dodecanal (95%, 87 g) as a solution in ethanol (20 mL) was metered continuously to the reaction mixture over the course of 6 h. The temperature (30° C.) and hydrogen pressure (5 bar) were maintained. After complete addition of the aldehyde, the reaction mixture was stirred at a temperature of 30° C. and a hydrogen pressure of 5 bar for 6 h, then cooled to room temperature and purged with nitrogen gas (thrice at 5 bar). Afterwards, the catalyst was filtered off (the filter cake was washed with ethanol) and the solvent was removed by evaporation, yielding the crude material as a solid.

### Example 37

Palladium on carbon black (10 wt.-%, 2 g) was added to a mixture of sarcosine (100%, 20 g) and dodecanal (95%, 87 g) in ethanol (70 mL) in an autoclave. The reaction vessel was closed and subsequently purged with nitrogen gas (thrice at 5 bar) and hydrogen gas (thrice at 5 bar). At an initial hydrogen pressure of 5 bar, stirring (700 rpm) was applied. The reaction mixture was warmed to 30° C. The temperature (30° C.) and hydrogen pressure (5 bar) were maintained and the reaction mixture was stirred 12 h, then cooled to room temperature and purged with nitrogen gas (thrice at 5 bar). Afterwards, the catalyst was filtered off and the filter cake was washed twice with ethanol. The solvent was removed by evaporation, yielding the crude material as a solid.

TABLE 2

| | | Solvent | | | | | | | | | |
|---|---|---|---|---|---|---|---|---|---|---|---|
| | | Dilution | δ ¹ | Pd/C ² | Amino | | Time | H₂ | T | C₂₄ ³ | C₁₂ ⁴ |
| # | Solvent | Ratio ¹ | [MPa^{1/2}] | [g] | Compound | Aldehyde | [h] | [bara] | [° C.] | [%] | [%] |
| 34 | EtOH | 1.1 | 26.2 | 2.0 | L-proline | dodecanal | 6 + 6 ⁵ | 5 | 30 | 2.9 | 61 |
| | (90 mL) | | | | (20 g) | (66 g) | | | | | |
| 35 * | EtOH | 1.1 | 26.2 | 2.0 | L-proline | dodecanal | 12 | 5 | 30 | 67 | 29 |
| | (90 mL) | | | | (20 g) | (66 g) | | | | | |
| 36 | EtOH | 0.7 | 26.2 | 2.0 | sarcosine | dodecanal | 6 + 6 ⁵ | 5 | 30 | 3.5 | 56 |
| | (70 mL) | | | | (20 g) | (87 g) | | | | | |
| 37 * | EtOH | 0.7 | 26.2 | 2.0 | sarcosine | dodecanal | 12 | 5 | 30 | 59 | 36 |
| | (70 mL) | | | | (20 g) | (87 g) | | | | | |

¹ Hildebrandt solubility parameter
² 10 wt.-% palladium on carbon black (no water content)
³ proportion of surfactant having a C₂₄ chain found in crude material product
⁴ proportion of surfactant having a C₁₂ chain found in crude material product
⁵ time for continuous dosing of the aldehyde, plus additional stirring time
* comparative example

In Examples 34 and 36, the process was conducted in feed operation, wherein dodecanal was continuously dosed into the solution of the amino compound. In Comparative Examples 35 and 37, on the other hand, the amino compound and dodecanal were provided as a mixture.

The proportions of surfactant having a C₁₂ chain and surfactant having a C₂₄ chain in the obtained crude material are shown in Table 2. The proportion of surfactant having a C₂₄ chain is indicative of the degree of aldol condensation of the aldehyde with itself to form the aldehyde dimer and subsequent reaction to form the C₂₄ sidechain surfactant. It is evident that the feed operation performed in Examples 34 and 36 largely suppresses the self-aldol condensation of the aldehyde, which allows for higher selectivity and moreover suppresses the excessive formation of dimer-based surfactants with less advantageous properties.

### Example 38

The reaction product of L-proline and dodecanal as obtained in Example 33b was analyzed and found to comprise less than 0.1 wt.-% of a proline-based dimeric surfactant with a C₂₄ chain derived from the self-aldol condensation product of dodecanal, namely (2-decyltetradecyl)proline.

3.0 parts by weight of the product obtained in Example 35 were mixed with 97 parts by weight of the product obtained Example 33b to obtain a surfactant composition comprising approximately 2 wt.-% of the proline-based dimeric surfactant with a C₂₄ chain.

7.5 parts by weight of the product obtained in Example 35 were mixed with 92.5 parts by weight of the product obtained in Example 33b to obtain a surfactant composition comprising approximately 5 wt.-% of the proline-based dimeric surfactant with a C₂₄ chain.

44.7 parts by weight of the product obtained in Example 35 were mixed with 55.3 parts by weight of the product obtained in Example 33b to obtain a surfactant composition comprising approximately 30 wt.-% of the proline-based dimeric surfactant with a C₂₄ chain.

The product of Example 33b and the obtained mixtures with the proline-based dimeric surfactant with a C₂₄ chain were subjected to surfactant tests. The results are shown in Table 3.

TABLE 3

| Content of | <0.1 | 2 | 5 | 30 |
|---|---|---|---|---|
| (2-decyltetradecyl)proline | wt.-% | wt.-% | wt.-% | wt.-% |
| Foam Volume [mL] ¹ | 650 | 730 | 630 | 50 |
| Cotton Wetting | 28 | 21 | 37 | 280 |
| Time [s] ² | | | | |
| Dynamic Surface | 38.3 | 36.7 | 38.7 | 66.4 |
| Tension [mN/m] ³ | | | | |
| Surface Tension vs. | 3.13 | 1.57 | 2.17 | 5.42 |
| Hexadecane [mN/m] ⁴ | | | | |
| Contact Angle [°] ⁵ | 65.8 | 59.8 | 59.2 | 82.7 |

¹ at 2 g/L in tap water with a water hardness of 10°d, measured according to EN 12728
² at 1 g/L in deionized water with 10 wt.-% of Na₂CO₃, measured according to EN 1772
³ at 1 g/L in deionized water, measured at 23° C. after 0.10 s
⁴ at 1 g/L in deionized water, measured at 23° C. after 10 min
⁵ at 0.2 g/L in deionized water, measured on polyethylene at 40° C. after 10.0 s

The reaction product of sarcosine and dodecanal obtained in Example 25 was analyzed and found to comprise less than 0.22 wt.-% of a sarcosine-based dimeric surfactant with a C₂₄ chain derived from the self-aldol condensation product of dodecanal, namely N-(2-decyltetradecyl)-N-methylglycine.

3.4 parts by weight of the product obtained in Example 37 were mixed with 96.6 parts by weight of the product obtained Example 25 to obtain a surfactant composition comprising approximately 2 wt.-% of the sarcosine-based dimeric surfactant with a C₂₄ chain.

8.5 parts by weight of the product obtained in Example 37 were mixed with 91.5 parts by weight of the product obtained in Example 25 to obtain a surfactant composition comprising approximately 5 wt.-% of the sarcosine-based dimeric surfactant with a C₂₄ chain.

50.8 parts by weight of the product obtained in Example 37 were mixed with 49.2 parts by weight of the product obtained in Example 25 to obtain a surfactant composition comprising approximately 30 wt.-% of the sarcosine-based dimeric surfactant with a C₂₄ chain.

The product of Example 25 and the obtained mixtures with the sarcosine-based dimeric surfactant with a C₂₄ chain were subjected to surfactant tests. The results are shown in Table 4.

TABLE 4

| Content of N-(2- | <0.22 | 2 | 5 | 30 |
|---|---|---|---|---|
| decyltetradecyl)-N-methylglycine | wt.-% | wt.-% | wt.-% | wt.-% |
| Foam Volume [mL] ¹ | 730 | 750 | 690 | 150 |
| Cotton Wetting Time [s] ² | 16 | 12 | 20 | 69 |
| Dynamic Surface Tension [mN/ | 51.8 | 47.2 | 52.8 | 53.1 |
| m] ³ | | | | |
| Surface Tension vs. Hexadecane | 3.74 | 2.74 | 3.84 | 4.47 |
| [mN/m] ⁴ | | | | |
| Contact Angle [°] ⁵ | 62.8 | 57.4 | 59.4 | 78.0 |

¹ at 2 g/L in tap water with a water hardness of 10°d, measured according to EN 12728
² at 1 g/L in deionized water with 10 wt.-% of Na₂CO₃, measured according to EN 1772
³ at 1 g/L in deionized water, measured at 23° C. after 0.10 s
⁴ at 1 g/L in deionized water, measured at 23° C. after 10 min
⁵ at 0.2 g/L in deionized water, measured on polyethylene at 40° C. after 10.0 s

1. A process for the production of a surfactant of general formula (I) 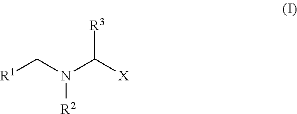
or a salt thereof, wherein
X is selected from the group consisting of COOH and CH₂SO₃H;
R¹ is selected from the group consisting of C₅-C₁₇-alkyl and C₅-C₁₇-alkenyl;
R² is selected from the group consisting of hydrogen, C₁-C₄-alkyl and CH₂PO₃H₂;
R³ is selected from the group consisting of hydrogen and C₁-C₈-alkyl, wherein C₁-C₈-alkyl is optionally substituted with one or more substituents selected from the group consisting of a hydroxy group, an amino group, a C₁-C₈-alkylamino group, a carboxyl group, a C₁-C₈-alkylcarboxylate group, a C₁-C₈-alkylamido group, a thio group, a C₁-C₈-alkylthio group, a guanidino group, and an aromatic group optionally substituted with a hydroxy group;
or wherein R² and R³, together with the nitrogen atom to which R² is bound and the carbon atom to which R³ is bound, form a 5- or 6-membered heterocycle, optionally substituted with a hydroxy group,
the process comprising reductive amination of an aldehyde of general formula (II) 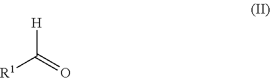
wherein R¹ is defined as above;
with an amino compound of general formula (III) 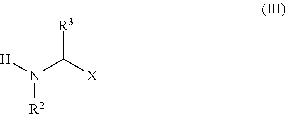
or a salt thereof, wherein
R², R³ and X are defined as above;
in the presence of molecular hydrogen and a heterogeneous catalyst comprising a group 10 element of the periodic table of the elements;
wherein the reductive amination is conducted in feed operation, wherein the amino compound of general formula (III) is provided, and the aldehyde of general formula (II) is metered thereto; and
wherein the reductive amination is performed
at a pressure of molecular hydrogen of at least 1 bara;
at a temperature of at least 25° C.; and
in a solvent having a Hildebrand solubility parameter δ in the range of 18 to 38 MPa^{1/2} at a solvent dilution ratio less than 7.0 L per kg, the solvent dilution ratio being the total volume of solvent to the total weight of the aldehyde of general formula (II) and the amino compound of general formula (III).

2. The process according to claim 1, wherein R¹ is C₇-C₁₇-alkyl, or wherein R¹ is C₉-C₁₇-alkenyl.

3. The process according to claim 1, wherein R² is C₁-C₃-alkyl and wherein R³ is hydrogen.

4. The process according to claim 1, wherein R² and R³, together with the nitrogen atom to which R² is bound and the carbon atom to which R³ is bound, form an optionally substituted pyrrolidinyl group or an optionally substituted piperidinyl group.

5. The process according to claim 1, wherein the heterogeneous catalyst is present in an amount of at most 2.5 wt.-%, calculated as group 10 element relative to the weight of the amino compound of general formula (III).

6. The process according to claim 1, wherein the group 10 element is selected from the group consisting of nickel, palladium and platinum.

7. The process according to claim 6, wherein the heterogeneous catalyst is palladium on activated carbon.

8. The process according to claim 1, wherein the solvent comprises a protic solvent and/or an aprotic solvent comprising an ether moiety.

9. The process according to claim 8, wherein the solvent is an aliphatic alcohol or a mixture of an aliphatic alcohol and water.

10. The process according to claim 9, wherein the solvent is a mixture of an aliphatic alcohol and water in a volume ratio in the range of 1:1 to 4:1.

11. The process according to claim 1, wherein the aldehyde of general formula (II) and/or the amino compound of general formula (III) are bio-based compounds.

12. A surfactant composition, comprising a surfactant of general formula (I) or a salt thereof as defined in claim 1 in an amount of at least 90 wt.-%, relative to the weight of the surfactant composition, and a compound of general formula (IV) 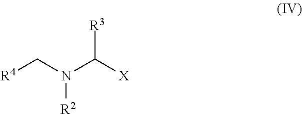
or a salt thereof in an amount of 0.01 to 5.0 wt.-%, relative to the weight of the surfactant composition,
wherein X, R² and R³ are defined as above; and R⁴ is selected from the group consisting of C₁₀-C₃₄-alkyl and C₁₀-C₃₄-alkenyl, with the proviso that the number of carbon atoms in R⁴ differs from the number of carbon atoms in R¹ of the surfactant of general formula (I).

13. A compound selected from the group consisting of
(2S,4R)-N-n-nonyl-4-hydroxypyrrolidine-2-carboxylic acid,
(2S,4R)-1-(2-ethylhexyl)-4-hydroxypyrrolidine-2-carboxylic acid,
(2S,4R)-1-(3,7-dimethyloctyl)-4-hydroxypyrrolidine-2-carboxylic acid,
(3,7-dimethyloctyl)-L-proline,
N-n-nonyl-N-(phosphonomethyl)glycine,
N-(2-ethylhexyl)-N-(phosphonomethyl)glycine,
N-(3,7-dimethyloctyl)-N-(phosphonomethyl)glycine,
1-(3,7-dimethyloctyl)piperidine-2-carboxylic acid,
N-(3,7-dimethyloctyl)-N-methylglycine,
2-((2-ethylhexyl)(methyl)amino)ethane-1-sulfonic acid,
2-((3,7-dimethyloctyl)(methyl)amino)ethane-1-sulfonic acid,
N-(2-ethylhexyl)-N-methylalanine,
N-(3,7-dimethyloctyl)-N-methylalanine,
N-n-nonyl-L-leucine,
N-n-nonyl-L-isoleucine,
2-(nonylmethylamino)ethanesulfonic acid; and
N-n-nonyl-L-glutamic acid.

14. A composition comprising at least one compound according to claim 13.

15. A method of using the compound according to claim 13, the method comprising using the compound as a surfactant.

16. A method of using the composition according to claim 14, the method comprising using the composition as a surfactant.

17. The process according to claim 1, wherein R¹ is C₇, C₈, or C₁₁-alkyl; or wherein R¹ is C₉-alkenyl.

18. The process according to claim 1, wherein R² is methyl, and wherein R³ is hydrogen.

19. The process according to claim 1, wherein the group 10 element is selected from the group consisting of nickel and palladium.

20. The process according to claim 1, wherein the solvent is a mixture of ethanol and water in a volume ratio in the range of 1:1 to 4:1.
